(19) United States
(12) Patent Application Publication
(10) Pub. No.: US 2007/0068521 A1
(43) Pub. Date: Mar. 29, 2007

(54) Portable oxygen generating system and method of using the same

(75) Inventors: Jui-Chi Wang, Taipei (TW); Wen-Feng Yang, Hsinchu (TW); Shiow-Chen Wang, Taipei (TW)

Correspondence Address: BAKER & MCKENZIE LLP;PATENT DEPARTMENT, 2001 ROSS AVENUE, SUITE 2300, DALLAS, TX, 75201, US

(73) Assignee: TreatYou Medical Technology Co.,

(21) Appl. No.: 11/236,228

(22) Filed: Sep. 27, 2005

(51) Int. Cl. B01J 7/00; A62B 7/08; A61L 9/00

(52) U.S. Cl. 128/202.26; 422/305; 422/236; 128/200.24

(57) ABSTRACT

A portable oxygen generating system comprises a reaction chamber having at least a first storage compartment for containing a first reacting substance and a second storage compartment for containing a second reacting substance, a dispensing mechanism operable to cause the first and second reacting substances to contact with each other so as to create a chemical reaction to produce an oxygen gas, and a gas outlet for delivering the produced oxygen gas. To use the system, the user pushes on a switch to initiate the production of oxygen.

### FIELD OF THE INVENTION

The present invention relates to the construction of a portable oxygen generating system and a method of using the same.

### DESCRIPTION OF THE RELATED ART

A portable oxygen supply system may save lives in hostile environments, such as a building on fire, where the ambient air may contain contaminates or noxious substances harmful to a human respiratory system. In these situations of emergency where the ambient air is not breathable, the use of a portable oxygen supply can prevent suffocation while waiting for medical rescues. In other therapeutic or medical applications, the oxygen supply system may also be used as an auxiliary oxygen source that assists the user's lungs to get enough amounts of oxygen to the blood.

A conventional oxygen supply system is an oxygen tank or cylinder that stores oxygen under pressure. To get access to the oxygen contained in the tank, the user opens a valve connected to a gas outlet of the oxygen tank, and the oxygen then can be breathed through a respiratory mask connected to the oxygen tank. A disadvantage of the oxygen tank is that since it stores oxygen under pressure, the tank may become hazardous if it is inadvertently dropped. Further, the content of the tank evaporates, which requires regular inspection and refill operations from a service technician.

An oxygen generating system is another type of oxygen supply system known in the art, which is usually constructed from a bottle enclosing a reaction chamber in which reacting substances are put in contact with each other to chemically react and produce oxygen. U.S. Pat. No. 4,508,700 to Hoshiko, the disclosure of which is incorporated herein by reference, describes a method of producing oxygen by putting a solid substance containing peroxide chemicals in contact with water. The peroxide substance is usually in a powder form contained in a packet. To produce oxygen, a user opens the packet, drops it with the peroxide powder substance in the reaction chamber previously filled with water, and then hermetically closes the bottle. The chemical reaction for producing oxygen then takes place inside the reaction chamber, and the produced oxygen passes through a filter system before it is delivered through a respiratory mask for a human's breathing. One disadvantage of such a system implementation is that it requires many manual operations from the user, which may not always be appropriately followed in every emergency situation. Further, the reaction occurs with an irregular oxygen flow rate.

U.S. Pat. No. 6,123,069 to Davis, the disclosure of which is also incorporated herein by reference, describes another oxygen generating system. In this system implementation, a sodium perborate anhydrous is initially placed in a chemical container of the oxygen generating system. To use the system, a user fills a second container placed over the chemical container with water, and then places a respiratory mask over the nose and mouth to breath oxygen. The water contained in the second container progressively flows down into the chemical container to react with the sodium perborate anhydrous and produce oxygen. One disadvantage of this system is that the user has to get access to a water source required to initiate the chemical reaction, which may not be possible in every situation, and even less likely in emergency situations. Another disadvantage is that the oxygen flow rate is irregular as the chemical reaction progressively consumes the reacting chemicals.

Therefore, there is presently a need for an improved oxygen generating system that is portable, can be simply and promptly triggered to initiate the production of oxygen, and allows a timely control to the production of oxygen so that it can be delivered with a uniform flow rate for the user breathing.

### SUMMARY OF THE INVENTION

The application describes a portable oxygen generating system and a method of using the same.

In one embodiment, a portable oxygen generating system comprises a reaction chamber having at least a first storage compartment for containing a first reacting substance and a second storage compartment for containing a second reacting substance, a dispensing mechanism operable to cause the first and second reacting substances to contact with each other so as to create a chemical reaction to produce an oxygen gas, and a gas outlet for delivering the produced oxygen gas.

In one embodiment, the dispensing mechanism includes a movable element operable to break an area of one of the first or second storage compartment. In some embodiments, the movable element is actuated via a spring element. In some embodiments, the movable element includes a rotary rod with a cutting tip. In other embodiments, the movable element includes a sliding punch end.

In some variant embodiments, the dispensing mechanism comprises a movable plate operable to open one of the first or second storage compartment. In some embodiments, the movable plate is actuated via a spring element. In some variant embodiments, the dispensing mechanism includes a switch button and is configured to actuate upon the application of a force on the switch button for releasing the second reacting substance from the second storage compartment.

In some variations, the second storage compartment includes a plurality of isolated partitions respectively containing doses of the second reacting substance. In some implementations, the dispensing mechanism is configured to sequentially open the partitions. In other implementations, the dispensing mechanism includes a timer configured to timely control the opening of the partitions. In some embodiments, the oxygen generating system comprises a filter system configured to filter the oxygen gas produced in the reaction chamber.

In some embodiments, the oxygen generating system comprises a buffer bag connected to the gas outlet, and a respiratory mask connected to the buffer bag. In some variations, the buffer bag includes a first buffer chamber connected with the gas outlet, a second buffer chamber connected with the respiratory mask, and a separator wall including at least a hole through which the first buffer chamber communicates with the second buffer chamber. In some variations, the buffer bag is connected to a carrying strap. In other variations, the buffer bag is configured to accommodate the reaction chamber therein.

The application also describes a method of using a portable oxygen generating system. In one embodiment, the method comprises providing an oxygen generating system having a switch, and switching on the oxygen generating system to trigger a chemical reaction to produce an oxygen gas. In some embodiments, the method includes breathing the oxygen gas via a respiratory mask connected to a buffer bag. In other embodiments, switching on the oxygen generating system includes pushing on a switch button provided on the oxygen generating system.

The foregoing is a summary and shall not be construed to limit the scope of the claims. The operations and structures disclosed herein may be implemented in a number of ways, and such changes and modifications may be made without departing from this invention and its broader aspects. Other aspects, inventive features, and advantages of the invention, as defined solely by the claims, are described in the non-limiting detailed description set forth below.

### BRIEF DESCRIPTION OF THE DRAWINGS

FIG. 1A is a schematic view of a portable oxygen generating system according to an embodiment of the invention;

FIG. 1B is a schematic view of a portable oxygen generating system switched to initiate a chemical reaction for producing oxygen according to an embodiment of the invention;

FIG. 1C is a schematic view illustrating a gas flowing path inside a portable oxygen generating system according to an embodiment of the invention;

FIG. 2A is a cross-sectional view of a dispensing mechanism suitable for use with a portable oxygen generating system according to an embodiment of the invention;

FIG. 2B is a top view of the mechanism shown in FIG. 2A;

FIG. 3A is a cross-sectional view of a dispensing mechanism suitable for use with a portable oxygen generating system according to another embodiment of the invention;

FIG. 3B is a top view of the mechanism shown in FIG. 3A;

FIG. 4A is a cross-sectional view of a dispensing mechanism suitable for use with a portable oxygen generating system according to another variant embodiment of the invention;

FIG. 4B is a top view of the mechanism shown in FIG. 4A;

FIG. 5A is a schematic view of a portable oxygen generating system according to another embodiment of the invention;

FIG. 5B is a schematic view of a portable oxygen generating system switched to initiate a chemical reaction for producing oxygen according to an embodiment of the invention;

FIG. 5C is a schematic view illustrating a gas flowing path inside a portable oxygen generating system according to another embodiment of the invention;

FIG. 6 is a schematic view of a portable oxygen generating system used with a buffer bag according to an embodiment of the invention;

FIG. 7 is a schematic view of a buffer bag suitable for use with a portable oxygen generating system according to another embodiment of the invention;

FIG. 8A is a schematic view of another buffer bag implementation suitable for accommodating a portable oxygen generating system according to an embodiment of the invention;

FIG. 8B illustrates a buffer bag implementation with a double-chamber structure suitable for use with a portable oxygen generating system according to an embodiment of the invention; and

FIG. 8C is a schematic view illustrating a gas flowing path inside the buffer bag of FIG. 8B.

### DETAILED DESCRIPTION OF THE EMBODIMENTS

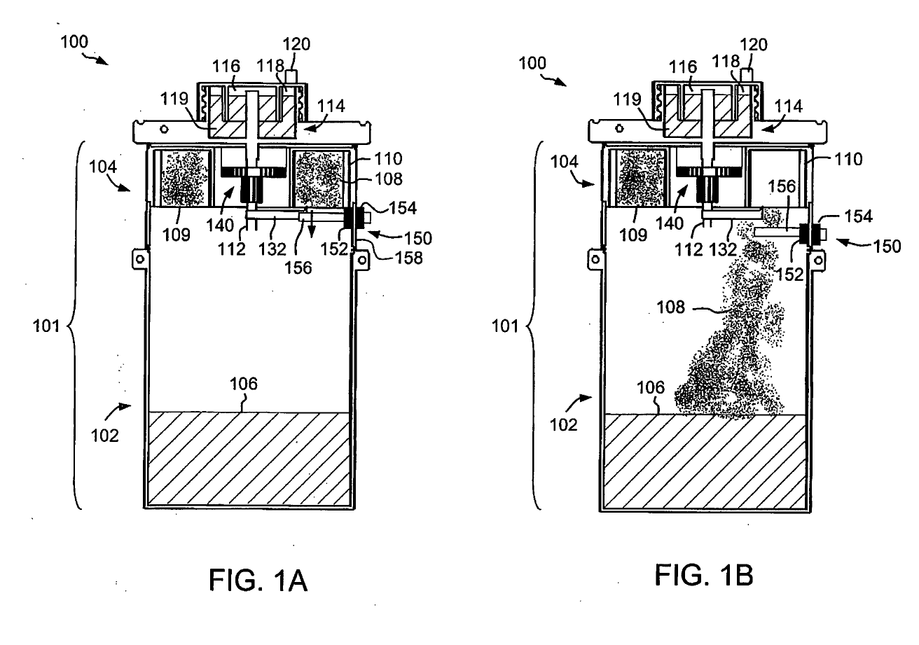

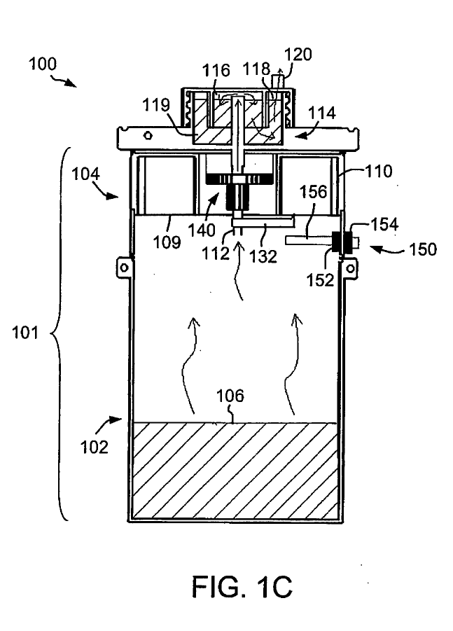

Reference is made to FIGS. 1A through 1C to describe a portable oxygen generating system according to an embodiment of the invention. Reference numeral 100 generally designates the oxygen generating system, which includes a reaction chamber 101 of an approximately cylindrical shape. The reaction chamber 101 includes a first storage compartment 102 and a second storage compartment 104 separated from each other. The first storage compartment 102 contains a first reacting substance 106, which can be water in the present embodiment. The second storage compartment 104 contains another reacting substance 108, which can be a peroxide powder substance in the present embodiment.

A pipe 112 connects the reaction chamber 101 with a first chamber 116 of a filter system 114, while a second chamber 118 of the filter system 114 communicates with a supply outlet 120. The first and second chambers 116 and 118 of the filter system 114 are isolated and separated from each other via a filtration medium 119, which can be water in one embodiment of the invention. The filter system 114 filters and moisturizes an oxygen gas produced during a chemical reaction between the first and second reacting substances 106 and 108 occurring inside the reaction chamber 101. The chemical reaction for producing oxygen is initiated and controlled by adequately putting the first and second reacting substances 106 and 108 in contact with each other through a dispensing mechanism described below.

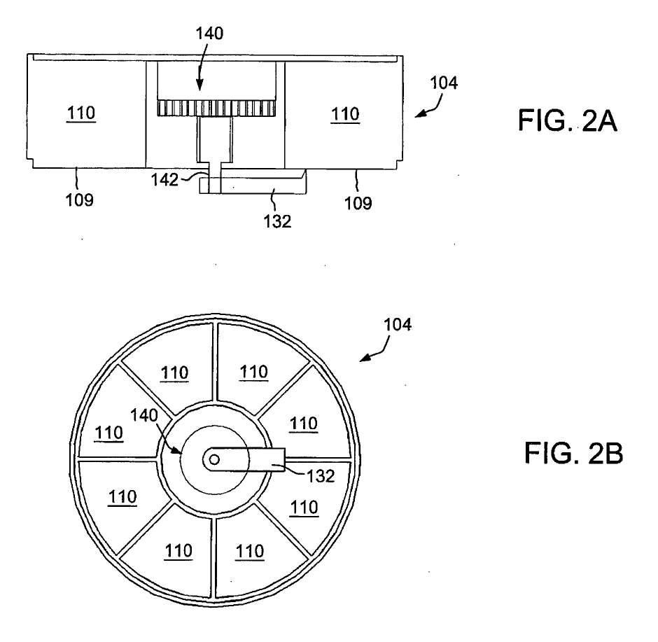

FIGS. 2A and 2B are enlarged views of a dispensing mechanism configured to timely release the second reacting substance contained in the second storage compartment 104 according to an embodiment of the invention. The second storage compartment 104 has an approximately annular shape, and is divided into isolated partitions 110 respectively containing specific doses of the second reacting substance. A bottom surface 109 of each partition 110 is configured so as to selectively open and release a dose of the second reacting substance into the reaction chamber.

In the embodiment of FIGS. 2A-2B, a rotary rod 132 having a cutting tip rotates in a parallel plane to perforate and cut the bottom surface 109, and thereby open each partition 110. Plastic film or other adequate materials may be suitable for the bottom surface 109 of each partition 110. In this embodiment, a spring mechanism such as a mechanical timer 140 can be assembled to drive the rotation of the rotary rod 132 via an axle 142. The mechanical timer 140 can be configured to timely rotate the rod 132 a specific angle to cut one or more partition 110 at each actuation of the rod 132. The doses of the second reacting substance in the partitions 110 thus can be timely delivered in controlled amounts so as to produce oxygen with a uniform flow rate.

Referring to FIGS. 1A through 1C, a switch mechanism 150 is provided to allow the user to mechanically trigger the dispensing mechanism and initiate the chemical reaction to produce oxygen according to an embodiment of the invention. In the illustrated construction, the switch mechanism 150 includes a pair of inner and outer magnets 152 and 154 attracting each other at opposite sides of a wall 158, and a locking beam 156 connected to the inner magnet 152. The outer magnet 154 may take the form of a button operable by a user. The locking beam 156 is mounted so as to lock and prevent any movement of the rotary arm 132 in an initial state.

As shown in FIG. 1B, when the user slides down the outer magnet 154, the inner magnet 152 accordingly slides downward to disengage the locking beam 156. Subject to the action from the programmed timer 140, the rotary rod 132 then performs the controlled angular rotations as discussed above to sequentially cut each partition 110 for timely releasing the second reacting substance 108 into the reaction chamber 101. By a simple and short switching action, the user thus can promptly initiate the chemical reaction to produce oxygen.

Referring to FIG. 1C, the oxygen gas produced by the chemical reaction inside the reaction chamber 101 flows out through the pipe 112, and is filtered and moisturized via the filtration medium 119 before the gas is delivered for breathing through the supply outlet 120.

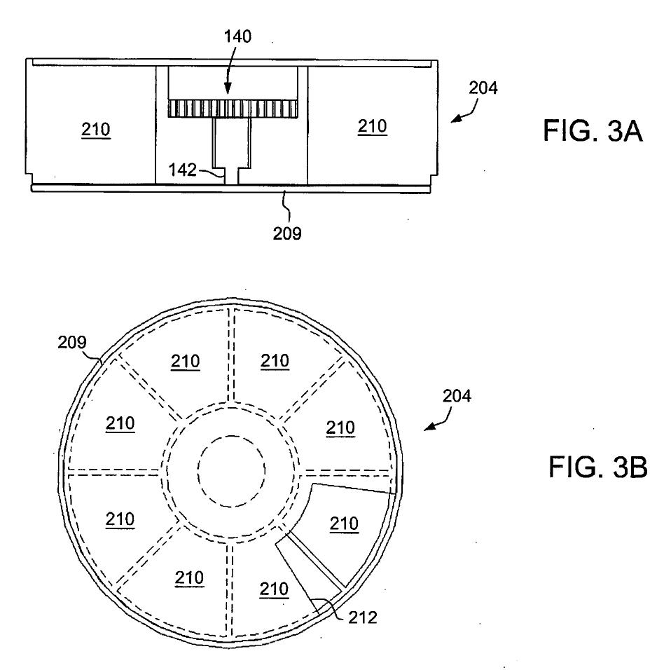

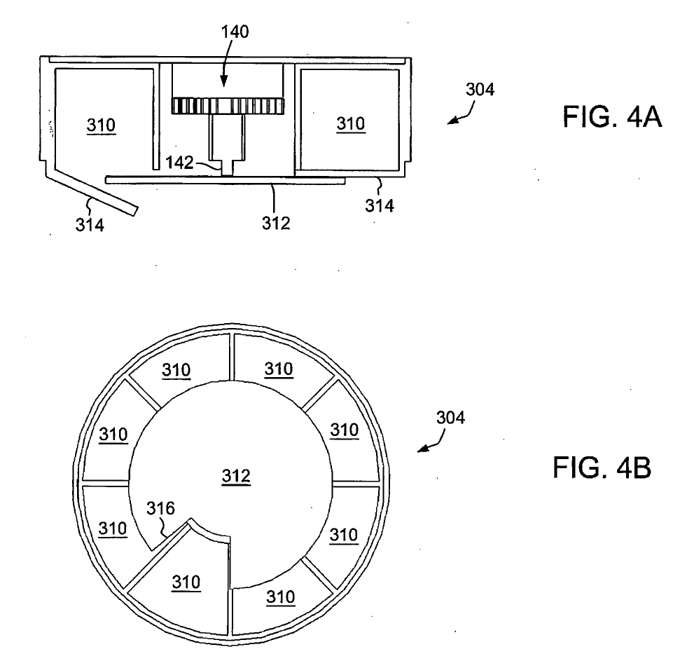

Many variant embodiments may be implemented to form a timely dispensing mechanism suitable for use with a portable oxygen generating system according to the invention. FIGS. 3A-3B and 4A-4B schematically illustrate two variant constructions of a timely dispensing mechanism according to the invention.

In FIGS. 3A and 3B, the second storage compartment 204 is divided into a plurality of isolated partitions 210 respectively having an open bottom obturated via a rotary diaphragm 209. The rotary diaphragm 209 has an opening recess 212 and is connected via the axle 142 to the mechanical timer 140. The opening recess 212 is sized so as to selectively open one or more partition 210 to release the second reacting substance when the diaphragm 209 rotates. In an initial state, the opening recess 212 of the diaphragm 209 may be positioned vis-á-vis an empty partition 210 with an edge of the recess 212 locked in abutment against the locking beam (not shown), while the other partitions 210 are filled with a reacting substance to be sequentially released once the rotation of the diaphragm 209 is initiated.

In FIGS. 4A and 4B, the second storage compartment 304 is divided into a plurality of isolated partitions 310 respectively having a bottom flap 314 movable to open each partition 310. In one embodiment, the bottom flaps 314 are exemplary made of a resiliently deflectable material. A rotary plate 312 having an opening recess 316 is connected via the axle 142 to the mechanical timer 140, and is placed in abutment against the bottom flaps 314 to close the partitions 310. When the rotary plate 312 rotates, the opening recess 316 creates a clearance that allows the resilient deflection of one or more flap 314 to selectively open one or more partition 310, and thereby release the contained reacting substance.

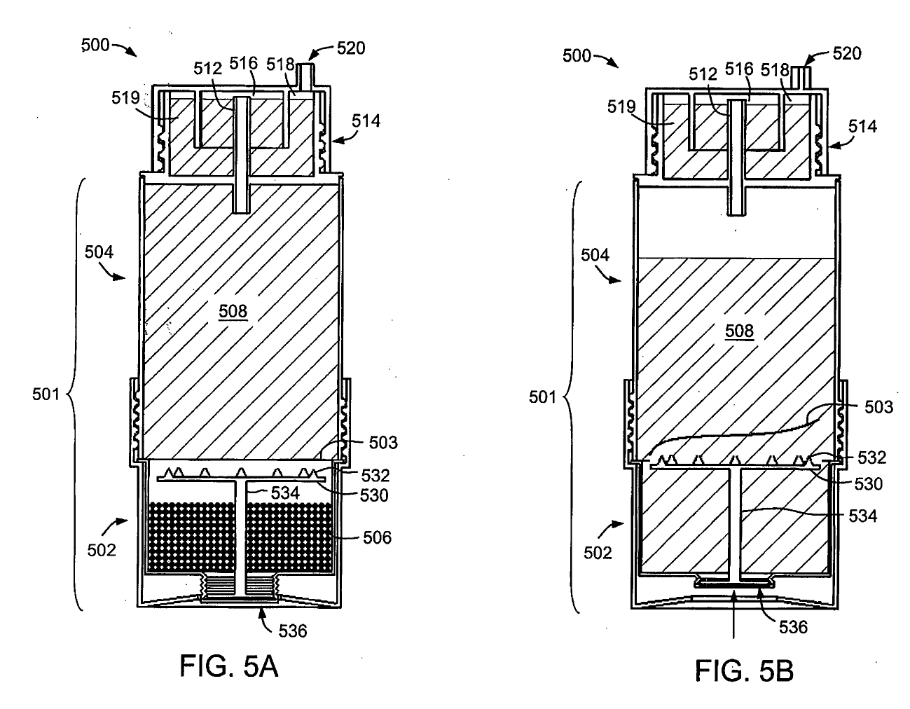

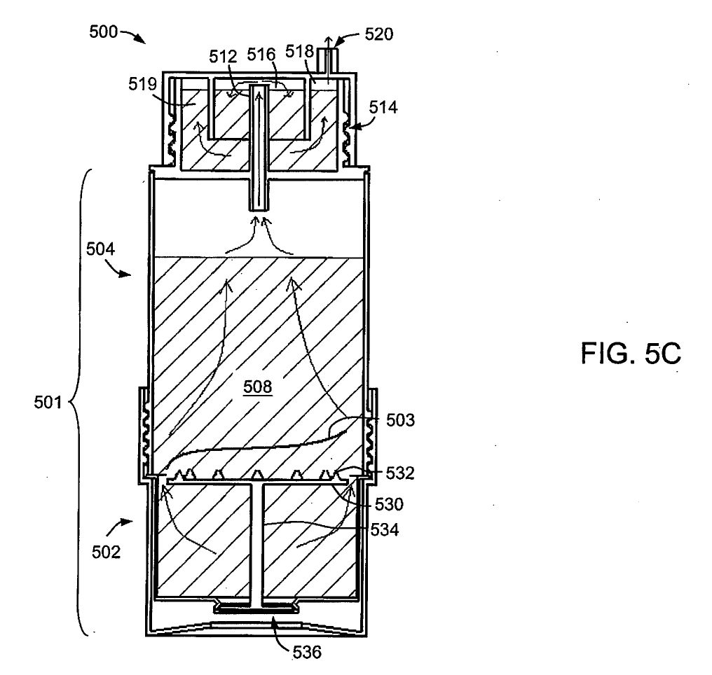

FIGS. 5A through 5C are schematic views of a portable oxygen generating system according to another embodiment of the invention. Reference numeral 500 generally designates the oxygen generating system, which includes a reaction chamber 501 of an approximately cylindrical shape. The reaction chamber 501 includes two storage compartments 502 and 504 separated from each other via an isolating barrier 503. The first storage compartment 502 contains a first reacting substance 506, and the second storage compartment 504 contains a second reacting substance 508. In one embodiment, the first reacting substance 506 may be a peroxide powder substance, and the second reacting substance 508 may be water. In one example of construction, the first and second storage compartments 502 and 504 may have surface areas provided with threaded portions so as to screw and mount with each other.

Referring to FIGS. 5A through 5C, a pipe 512 connects the reaction chamber 501 with a first chamber 516 of a filter system 514, while a second chamber 518 of the filter system 514 communicates with a supply outlet 520. The first and second chambers 516 and 518 of the filter system 514 are isolated from each other by a filtration medium 519, which can be water in one embodiment of the invention.

The oxygen generating system 500 also includes sliding rod 534 that terminates in a punch plate 530 at one first end, and connects with a push button 536 at an end opposite the punch plate 530. The punch plate 530 is provided with projecting tooth 532, and the push button 536 protrudes outward to be operable by a user. In an initial position, the punch plate 530 is located away from the isolating barrier 503.

As shown in FIG. 5B, when the user pushes the button 536, the exerted force accordingly causes the rod 534 to slide and urge the punch plate 530 against the isolating barrier 503 and break the barrier 503 so that the first and second storage compartments 502 and 504 communicate with each other. As a result, the first and second reacting substances 506 and 508 contact with each other to chemically react and produce an oxygen gas.

Referring to FIG. 5C, the oxygen gas produced by the chemical reaction inside the reaction chamber 501 flows out through the pipe 512, and is filtered and moisturized via the filtration medium 519 before it is delivered for breathing through the supply outlet 520.

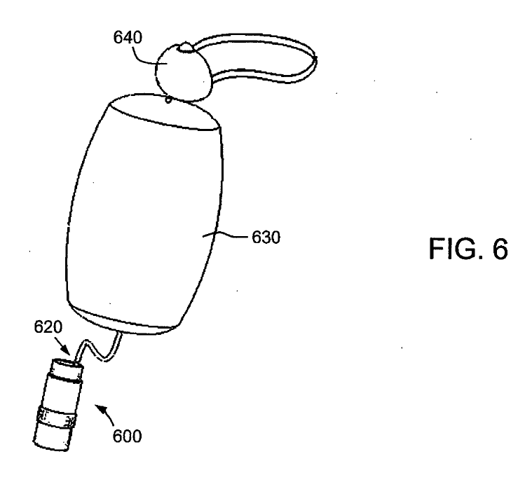

The foregoing description illustrates various embodiments of a portable oxygen generating system that includes a switching mechanism allowing a user to trigger the chemical reaction to produce oxygen. In one embodiment illustrated in FIG. 6, the oxygen gas produced from the portable oxygen generating system 600 then may be supplied to the user via a buffer bag 630 and a respiratory mask 640. One function of the buffer bag 630 is to bring the oxygen gas delivered at an outlet 620 of the oxygen generating system 600 to a pressure suitable for a smooth breathing. Another function of the buffer bag 630 is to mix the substantially pure oxygen gas produced at the outlet 620 with other gaseous elements, which may be an air gas mixture, so that the oxygen breathed at the user end through the respiratory mask 640 is less concentrated to prevent any overdose intoxication.

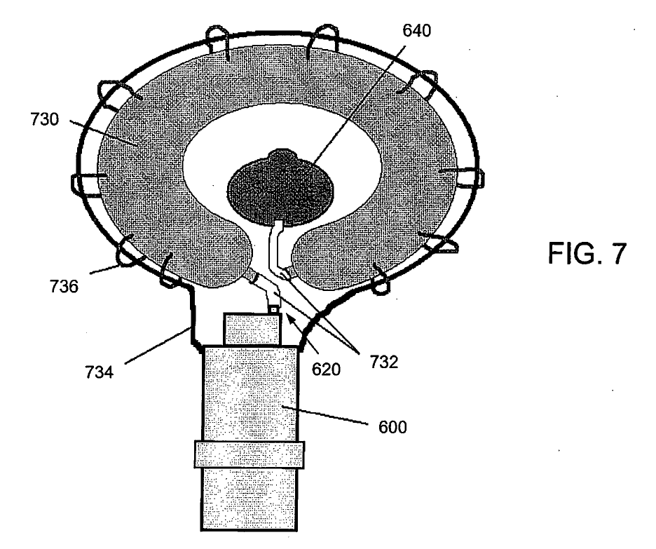

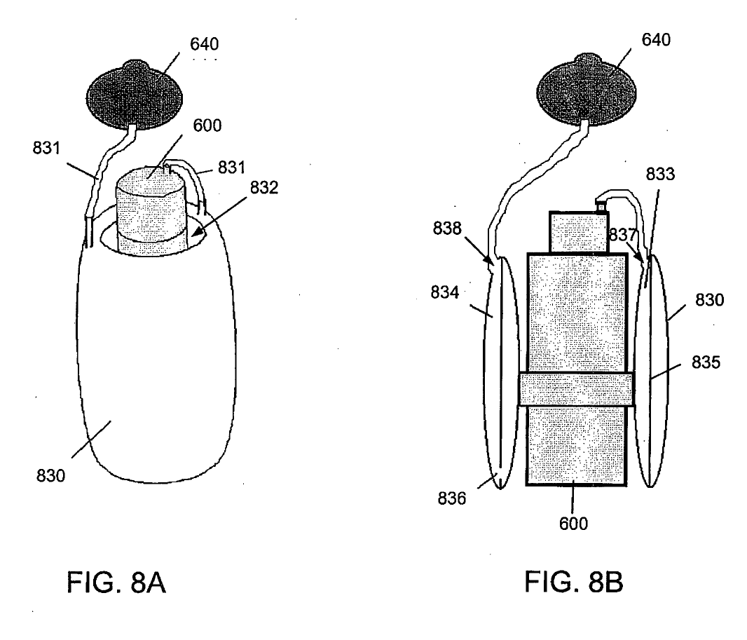

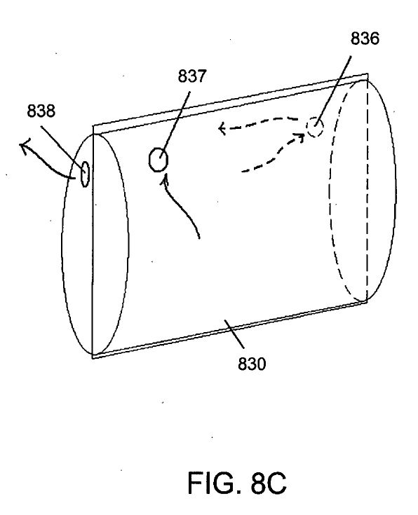

FIG. 7 and FIGS. 8A-8C are schematic views of possible variant embodiments of a buffer bag implementation suitable for use with a portable oxygen generating system according to the invention. In the embodiment of FIG. 7, the buffer bag 730 connects via joints 732 with the outlet 620 of the oxygen generating system 600 and the respiratory mask 640, respectively. A strap 734 is secured with the oxygen generating system 600 and the buffer bag 730 via buckles 736. Alternatively, the strap may be integrally incorporated with an edge of the buffer bag (not shown). The user can carry the oxygen generating system 600 with the strap 734 so that less stress charges are applied to the joints 732.

In FIG. 8A, the buffer bag implementation 830 may have an enveloping form with a cavity 832 that can accommodate the reaction chamber of the oxygen generating system 600 inside. The buffer bag 830 connects between the oxygen generating system 600 and the respiratory mask 640 via tubes 831. FIG. 8B illustrates a buffer bag implementation with a double-chamber structure according to an embodiment of the invention. As shown in FIG. 8B, the buffer bag 830 may include an inner chamber 833 and an outer chamber 834 substantially isolated from each other via a wall 835 except at a communicating hole 836 formed through the wall 835.

FIG. 8C illustrates the gas flowing path inside the buffer bag of FIG. 8B. As shown, the oxygen gas produced from the oxygen generating system flows in the inner chamber 833 of the buffer bag 830 via an inlet 837, travels through the communicating hole 836 into the outer chamber 834, and exits the buffer bag 830 via the outlet 838 to be breathed at the respiratory mask 640. The double-chamber structure of the buffer bag can advantageously prevent the chemical liquid inside the reaction chamber from flowing out to the respiratory mask.

It should be understood that the foregoing embodiments have been described only for the purpose of illustration, and many variations and modifications may be possible. For example, the portable oxygen generating system according to the invention may be used either with or without a buffer bag. Further, the portable oxygen generating system may be implemented either in a disposable form for a single use, or in a reusable form.

In a reusable implementation, one of the storage compartments containing, for example, a peroxide powder substance may be provided as a repairable and/or replaceable cartridge inside the portable oxygen generating system. The user thus can reuse the oxygen generating system by refilling clean water in the storage compartment and replacing the consumed and empty cartridge with a new one.

Realizations in accordance with the present invention therefore have been described in the context of particular embodiments. These embodiments are meant to be illustrative and not limiting. Many variations, modifications, additions, and improvements are possible. Accordingly, plural instances may be provided for components described herein as a single instance. Structures and functionality presented as discrete components in the exemplary configurations may be implemented as a combined structure or component. These and other variations, modifications, additions, and improvements may fall within the scope of the invention as defined in the claims that follow.

What is claimed is:

1. A portable oxygen generating system, comprising:
a reaction chamber, including at least a first storage compartment for containing a first reacting substance and a second storage compartment for containing a second reacting substance;
a dispensing mechanism operable to cause the first and second reacting substances to contact with each other so as to create a chemical reaction to produce an oxygen gas; and
a gas outlet for delivering the oxygen gas.

2. The system according to claim 1, wherein the dispensing mechanism includes a movable element operable to break an area of one of the first or second storage compartment.

3. The system according to claim 2, wherein the movable element is actuated via a spring element.

4. The system according to claim 2, wherein the movable element includes a rotary rod with a cutting tip.

5. The system according to claim 2, wherein the movable element includes a sliding punch end.

6. The system according to claim 1, wherein the dispensing mechanism comprises a movable plate operable to open one of the first or second storage compartment.

7. The system according to claim 6, wherein the movable plate is actuated via a spring element.

8. The system according to claim 1, wherein the dispensing mechanism is connected to a switch button and is configured to actuate upon the application of a force on the switch button to release the second reacting substance from the second storage compartment.

9. The system according to claim 1, wherein the second storage compartment includes a plurality of isolated partitions respectively containing doses of the second reacting substance.

10. The system according to claim 9, wherein the dispensing mechanism is configured to sequentially open the partitions.

11. The system according to claim 10, wherein the dispensing mechanism includes a timer configured to timely control the opening of the partitions.

12. The system according to claim 1, further comprising a filter system configured to filter an oxygen gas produced in the reaction chamber.

13. The system according to claim 1, further comprising:
a buffer bag connected to the gas outlet; and
a respiratory mask connected to the buffer bag.

14. The system according to claim 13, wherein the buffer bag includes:
a first buffer chamber connected with the gas outlet;
a second buffer chamber connected with the respiratory mask; and
a separator wall including at least an opening through which the first buffer chamber communicates with the second buffer chamber.

15. The system according to claim 13, wherein the buffer bag is connected to a carrying strap.

16. The system according to claim 13, wherein the buffer bag is configured to accommodate the reaction chamber therein.

17. The system according to claim 1, wherein one of the first or second reacting substance includes water.

18. The system according to claim 1, wherein one of the first or second storage compartment is repairable or replaceable.

19. A method of using a portable oxygen generating system, comprising:
providing an oxygen generating system having a switch; and
switching on the oxygen generating system to trigger a chemical reaction to produce an oxygen gas.

20. The method according to claim 19, further including breathing the oxygen gas via a respiratory mask connected to a buffer bag.

21. The method according to claim 19, wherein switching on the oxygen generating system to trigger a chemical reaction to produce an oxygen gas includes pushing on a switch button provided on the oxygen generating system.

22. A storage compartment for containing a reacting substance for use in a portable oxygen generating system, the system comprising a reaction chamber including the storage compartment, a dispensing mechanism operable to cause the reacting substance to react and produce an oxygen gas, and a gas outlet for delivering the oxygen gas, the storage compartment comprising a repairable or replaceable cartridge for reuse of the portable oxygen generating system.

23. A method of portably providing oxygen, comprising:
providing a reaction chamber, including at least a first storage compartment for containing a first reacting substance and a second storage compartment for containing a second reacting substance;
providing a dispensing mechanism operable to cause the first and second reacting substances to contact with each other so as to create a chemical reaction to produce an oxygen gas; and
providing a gas outlet for delivering the oxygen gas.
